(19) United States
(12) Patent Application Publication
(10) Pub. No.: US 2004/0091451 A1
(43) Pub. Date: May 13, 2004

(54) Biocompatible polymer for fixing biological ligands

(76) Inventors: Marie-Therese Charreyre, Lyon (FR); Franck D'Agosto, Dijon (FR); Arnaud Favier, Marsonnas (FR); Christian Pichot, Corbas (FR); Bernad Mandrand, Villeurbanne (FR)

Correspondence Address: Oliff & Berridge, PO Box 19928, Alexandria, VA, 22320, US

(21) Appl. No.: 10/296,889

(22) PCT Filed: May 29, 2001
(86) PCT No.: PCT/FR01/01663

(30) Foreign Application Priority Data
May 29, 2000 (FR) 00.06861

(51) Int. Cl. A61K 31/765; C08G 63/48; C08G 63/91

(52) U.S. Cl. 424/78.19; 525/54.2

(57) ABSTRACT

The invention concerns a biocompatible polymer having a mole weight more than 50000 g/mole, preferably 90000 g/mole for fixing biological ligands comprising at least a first linear segment consisting of a hydrophobic homopolymer derived from polymerisation of a hydrophobic monomer A; a second linear segment consisting of a hydrophilic polymer derived from copolymerisation of a monomer B bearing a reactive function X and a hydrophilic monomer C not bearing any reactive function, said second segment being covalently bound to one end of the first segment. The invention also concerns a biological polymer-ligand-conjugate, a device for capturing a target molecule comprising a solid support whereon is immobilised a biological polymer-ligand conjugate and methods for preparing said polymer. The invention is mainly applicable in the field of diagnosis.

The present invention relates to a biocompatible polymer for fixing biological ligands, to a biological polymer-ligand conjugate, to a device for capturing a target molecule comprising a solid support on which is immobilized a biological polymer-ligand conjugate, and also to methods for preparing the polymer.

Synthetic polymers have been used for a long time both in the therapeutic field for vectorizing active molecules or genes and in the diagnostic field. In the latter case, biological ligands are fixed to polymers either by complexation, by covalent bonding or by specific recognition, and the conjugates thus formed are used in tests for detecting target molecules essentially to increase the sensitivity. Thus, the Applicant has filed a certain number of patents relating to various polymers and their applications.

Patent FR 2 688 788 (Charles M. H. et al.) describes the synthesis and use of conjugates of biological ligands/copolymer based on maleic anhydride, for instance the maleic anhydride/methyl vinyl ether (AMVE) copolymer for fixing biological ligands to a solid support. Similarly, patent FR 2 707 010 (Mabilat C. et al.) describes a copolymer based on N-vinylpyrrolidone, for instance the N-vinylpyrrolidone/N-acryloxysuccinimide (NVPNAS) copolymer, again for fixing biological ligands to a solid support. These same copolymers have been used for signal amplification reactions (see patent FR 2 710 075, Mandrand B. et al.) or for the in situ synthesis of conjugates (see WO 99/07749, Minard C. et al.).

Although these various copolymers allow an improvement in sensitivity in diagnostic tests, they have a certain number of drawbacks:

The copolymer is adsorbed randomly onto the solid support. It is not known whether it is adsorbed via one or more biological ligands, or via segments of the polymer skeleton. In any case, the copolymer is adsorbed onto the solid support at several points distributed along the skeleton (loop mode). In this case, the availability of the biological ligands to react with target molecules is limited.

Furthermore, in certain cases, the conjugates have an aggregated structure (see, for example, Erout M. N. et al., Bioconjugate Chemistry, 7(5), 568-575, (1996) or Delair T. et al., Polymers for Advanced Technologies, 9, 349-361, (1998)). This aggregation phenomenon is entirely solved by the methods used in patent application WO 99/07749 but the sensitivity of the tests for detecting target molecules is found to be affected.

Another approach has been described by Ganachaud F. et al. in “Journal of Applied Polymer Science, 58, 1811-1824, 1995”. An N-vinylpyrrolidone homopolymer is functionalized at one end with biotin, thus allowing the oriented fixing of this homopolymer onto a surface functionalized with streptavidin to be envisioned.

This homopolymer is obtained by using an azo primer bearing two biotin functions. However, the use of such a bulky primer is reflected by: a low efficacy factor (this factor corresponds to the number of polymer chains formed from the decomposition of one primer molecule: in this case, this value is 0.17, whereas for standard primers the value is between 0.50 and 0.70); a very slow polymerization rate; and a final conversion of the monomer limited to 20.5%. Furthermore, the molar masses of the homopolymers obtained are very low (6000 g/mol), which the authors explain by the presence of many labile protons on this primer, promoting parasitic transfer reactions. Moreover, after fixing this homopolymer to a surface functionalized with streptavidin, no potential site remains for fixing the biological ligands to said homopolymer. Furthermore, no copolymer containing potential sites for fixing biological ligands has been synthesized by this approach.

If a copolymer had been synthesized according to the approach described in the previous paragraph, the low masses obtained would only make it possible to envision the fixing of 1 or 2 biological ligands per polymer chain. Furthermore, these biological ligands would be very close to the surface of the solid support and close together, which would be prejudicial to the reaction efficacy of these ligands with the target molecules.

In addition, modifying the solid surface with streptavidin in order to allow the attachment of the polymer-biological ligand conjugate modifies the nature of the surface in an uncontrolled manner depending on the structure of the protein.

An example of a polymer of low molar mass is given in patent U.S. Pat. No. 5,519,085, which relates to the stabilization of solid particles of pigment type in aqueous dispersions and which uses triblock polymers.

The molar mass of these polymers is less than 20,000 g/mol and is in fact in the range from 1000 to 7000 g/mol, which does not make it possible to achieve extensive grafting of biological molecules.

The present invention solves the problems mentioned above by proposing a novel type of polymer for fixing biological ligands, which has:

a controlled architecture to space the biological molecules for the solid support and promote the reactivity of these biological ligands with target molecules in solution,

a sufficient size to allow a high degree of grafting of the biological ligands while at the same time maintaining a space between said ligands and thus promoting the sensitivity of the diagnostic tests.

The methods for preparing these polymers by controlled polymerization techniques make it possible in principle to know the molar masses of the chains and to obtain them with low polydispersity indices, i.e. chains of very homogeneous size.

To this end, the present invention describes a biocompatible polymer with a molar mass of greater than 50,000 g/mol, preferably 90,000 g/mol, allowing the fixing of biological ligands, and comprising at least: a first linear segment consisting of a hydrophobic homopolymer resulting from the polymerization of a hydrophobic monomer A; a second linear segment consisting of a hydrophilic copolymer resulting from the copolymerization of a monomer B bearing a reactive function X and of a hydrophilic monomer C not bearing a reactive function, said second segment being covalently bonded to one end of the first segment and the two segments together constituting the skeleton of the polymer.

The expression “biocompatible polymer” means a polymer that does not disrupt the biological properties of the biological ligands fixed to the polymer in terms of molecular recognition.

The expression “biological ligand” means a compound that contains at least one recognition site allowing it to react with a target molecule of biological interest. Examples of biological ligands that may be mentioned include polynucleotides, antigens, antibodies, polypeptides, proteins and haptens.

The term “polynucleotide” means a sequence of at least two deoxyribonucleotides or ribonucleotides optionally comprising at least one modified nucleotide, for example at least one nucleotide comprising a modified base such as inosin, 5-methyldeoxycytidine, 5-dimethylaminodeoxyuridine, deoxyuridine, 2,6-diamino-purine, 5-bromodeoxyuridine or any other modified base allowing hybridization. This polynucleotide may also be modified in the internucleotide bond such as, for example, phosphorothioates, H-phosphonates or alkylphosphonates, in the skeleton such as, for example, α-oligonucleotides (FR 2 607 507) or PNAs (M. Egholm et al., J. Am. Chem. Soc., 114, 1895-1897, (1992) or 2′-O-alkylriboses. Each of these modifications may be taken in combination. The polynucleotide may be an oligonucleotide, a natural nucleic acid or its fragment, for instance a DNA, a ribosomal RNA, a messenger RNA, a transfer RNA or a nucleic acid obtained via an enzymatic amplification technique.

The term “polypeptide” means a sequence of at least two amino acids.

The term “amino acids” means the primary amino acids that encode proteins, the amino acids derived after enzymatic action, for instance trans-4-hydroxy-proline, and amino acids that are natural but not present in proteins, for instance norvaline, N-methyl-L-leucine and staline (see Hunt S. in Chemistry and Biochemistry of the amino acids, Barett G. C., ed., Chapman and Hall, London, 1985), amino acids protected with chemical functions that may be used in synthesis on a solid support or in liquid phase, and unnatural amino acids.

The term “hapten” denotes nonimmunogenic compounds, i.e. compounds that are incapable by themselves of promoting an immune reaction by producing antibodies, but are capable of being recognized by antibodies obtained by immunization of animals under known conditions, in particular by immunization with a hapten-protein conjugate. These compounds generally have a molecular mass of less than 3000 DA and usually less than 2000 DA and may be, for example, glycosylated peptides, metabolites, vitamins, hormones, prostaglandins, toxins or various medicinal products, nucleosides and nucleotides.

The term “antibody” includes polyclonal or monoclonal antibodies, antibodies obtained by genetic recombination, and antibody fragments such as Fab or F(ab′)₂ fragments. The term “antigen” denotes a compound capable of generating antibodies. The term “protein” includes holoproteins and heteroproteins, for instance nucleoproteins, lipoproteins, phosphoproteins, metalloproteins and glycoproteins which are either fibrous or globular in their characteristic conformational form.

The reference technique for measuring the molar mass of a polymer, which is expressed in the present invention by Mₚₑₐₖ (molar mass of the majority population of the polymer chains in g/mol), is steric exclusion chromatography coupled to a light-scattering detector (SEC/LSD). By determining by measurement the value of the refractive index increment (dn/dc) of the polymer under consideration in a suitable solvent, which solvent will be used as eluent for the SEC, the LSD detector gives “absolute” molar mass values as opposed to the molar mass values “relative” to a calibration (for example polystyrene standards in organic phase), when a conventional SEC technique is used.

The polymer skeleton consists of two linear segments, i.e. each monomer, with the exception of the ends, is linked to two other monomers sandwiching said monomer along the chain.

The first segment is a hydrophobic homopolymer, i.e. a polymer comprising a sequence of only one hydrophobic monomer A.

The second linear segment is a copolymer consisting of two monomers, the first monomer C providing hydrophilicity, in order to promote maximum deployment of the second segment in the aqueous phase, and the other monomer B providing a reactive function X in order to achieve either the covalent attachment of lateral segments, said lateral segments each having several potential sites for fixing biological ligands, or the direct fixing of biological ligands. Another role of the hydrophilic monomer is to space the sites of attachment of the side chain units or of the biological molecules.

The term “copolymer” should be understood as being a polymer formed from two different monomers B and C and especially random copolymers (in which the monomer units B and C are randomly distributed along the macromolecular chain) and alternating copolymers (in which the monomers B and C are regularly repeated in a general structure (BC)ₙ in which n is an integer). These various copolymers may be obtained by polycondensation reaction, or by free-radical, ionic or group-transfer chain polymerization, advantageously by live free-radical polymerization, by reversible termination polymerization (using nitroxide radicals), atom-transfer polymerization (ATRP), and preferably reversible addition-fragmentation chain transfer polymerization, known as RAFT (see WO 98/01478). These various polymerization techniques are described, for example, in K. Matyjazewski, Controlled Radical Polymerization, American Chemical Society Series, Washington D.C., U.S.A., 1997; G. Odian, Principles of Polymerization, Third edition, Wiley-Interscience Publication, 1991.

Preferably, the second segment is a random copolymer.

The monomer A is a hydrophobic monomer chosen from:

monomers of ethylene, propylene, vinylaromatic, acrylate, methacrylate, substituted acrylamide or methacrylamide derivative, styrene or substituted styrene derivative, vinyl halide (vinyl chloride), vinyl acetate or diene type or monomers containing nitrile functions (acrylonitrile).

The term “hydrophobic” means a monomer whose polymer has in aqueous phase a compact ball structure, corresponding to a Mark-Houwink-Sakurada coefficient (form factor) of less than 0.8. Preferably, the monomer A is chosen from methacrylate derivatives, acrylate derivatives and styrene derivatives, advantageously n-butyl acrylate, t-butyl acrylate and styrene.

The monomer B is a functional monomer, i.e. it can bear a reactive function X, chosen from functional monomers of acrylate, methacrylate, styrene, acrylamide and methacrylamide type, such as substituted acrylamide and methacrylamide derivatives, in particular polymerizable saccharide derivatives. Advantageously, B is N-acryloxysuccinimide, N-methacryloxysuccinimide, 2-hydroxyethyl methacrylate, 2-aminoethyl methacrylate, 2-hydroxyethyl acrylate, 2-aminoethyl acrylate, 1,2:3,4-di-O-isopropylidene-6-O-acryloyl-D-galactopyranose, and B is preferably N-acryloxysuccinimide, 2-aminoethyl acrylate or 1,2:3,4-di-O-isopropylidene-6-O-acryloyl-D-galactopyranose.

The reactive function X is chosen, for example, from amine, hydrazine, hydrozone, azide, isocyanate, isothiocyanate, alkoxyamine, aldehyde, epoxy, nitrile, maleimide, haloalkyl, hydroxyl or thiol groups or a carboxylic acid group activated in the form of the N-hydroxysuccinimide, pentachlorophenyl, trichlorophenyl, p-nitrophenyl or carboxyphenyl ester. Preferably, the reactive function X is chosen from amine and aldehyde functions or from carboxylic acid functions activated in the form of the N-hydroxysuccinimide ester.

The monomer C is a hydrophilic monomer comprising no reactive function. The term “hydrophilic” means a monomer whose polymer has in aqueous phase a deployed structure, corresponding to a Mark-Houwink-Sakurada coefficient of greater than 0.8. The monomer C is chosen from monomers derived from acrylamide, from methacrylamide or from N-vinylpyrrolidone. Preferably, the monomer C is N-vinylpyrrolidone (NVP) or N-acryloylmorpholine (NAM).

In the present invention, the first segment has a molar mass of between 10,000 and 250,000 g/mol to allow the immobilization on a solid support of the biocompatible polymer of the invention or of the polymer-biological ligand conjugate also according to the invention. The second segment has a molar mass of greater than 40,000 g/mol and preferably greater than 80,000 g/mol in order to have available a sufficient number of reactive functions X for fixing the biological ligands or the side segments.

Preferably, the second segment is a random polymer whose composition, expressed by the ratio of the amounts of monomers in moles: amount of monomer C to amount of monomer B, is between 1 and 10 and preferably between 1.5 and 4 to allow spacing of the reactive functions and thus to reduce the steric hindrance that might result from the coupling of the biological ligands or the side segments.

In another embodiment of the invention, the polymer also comprises at least one “side” segment, said side segment consisting of a linear homopolymer resulting from the polymerization of a monomer D bearing a reactive function Y (optionally protected) so as to achieve the fixing of biological ligands by covalent coupling, and said side segment being covalently bonded to the second segment of the skeleton at a single bonding point via reactive functions X. Advantageously, at least 10 side segments, more advantageously at least 30 side segments and preferably at least 70 side segments, are present on the polymer skeleton. When the polymer comprises at least one side segment, the polymer is said to be branched. The side segment is covalently bonded to the polymer skeleton at a single bonding point.

In one particular embodiment, the bond consists of a covalent bond between any reactive function Y of the monomer D of the side segment and a reactive function X of a monomer B of the skeleton.

In another particular preferential embodiment, to conserve a controlled architecture of the polymer (i.e. in order for the side segments to be fixed onto the skeleton in an oriented manner, forming a cylindrical space around the skeleton), the covalent bonding takes place between a reactive function other than Y present at the end of the side segment and a reactive function X of a monomer B of the skeleton. To this end, a technique of controlled polymerization, for instance living anionic polymerization or, preferably, living cationic polymerization, or a technique such as living free-radical polymerization, preferably the RAFT technique, is used to synthesize the side segment. This allows it to be functionalized at one end with a reactive function that is capable of reacting (complementary) with the function X of the monomer B. Between this reactive end function and the first unit of the homopolymer there is, in particular, a spacer arm of —(CH₂)ₙ-type with n being an integer greater than or equal to 1, in order to reduce the hindrance of the end of the side segment and to promote the reaction of this end with a function X of the skeleton. This also makes it possible to distance the reactive functions Y of the side segment from the skeleton.

The monomer D is chosen from functional monomers of the type such as acrylate, methacrylate, acrylamide, methacrylamide, vinyl ether, for instance chloroethyl vinyl ether (CEVE), polymerizable derivatives of a sugar, for instance glucose or galactose, advantageously from polymerizable galactose derivatives.

In particular, the monomer D, if it is a sugar derivative, may comprise a spacer arm of (CH₂)ₙO-type, with n being an integer greater than or equal to 1, between the sugar and the polymerizable function of the monomer D to distance the saccharide ring of the chain from the side segment and to improve the accessibility of the reactive function Y with respect to biological ligands. Preferably, the polymerizable function possibly borne by the spacer arm is introduced into position 6 of the saccharide ring for the same reasons. The secondary OH functions in positions 1, 2, 3 and 4 of the saccharide ring are protected in the form of acetate or benzoate, advantageously in the form of acetyl of cyclohexylidine type or preferably of isopropylidene type (and are deprotected after polymerization of the monomer D in order to achieve the covalent coupling of the biological ligands).

In particular, the monomer D is N-acryloxysuccinimide, N-methacryloxysuccinimide or 1,2:3,4-di-O-isopropylidene-6-O-acryloyl-D-galactopyranose. Preferably, the monomer D is 1,2:3,4-di-O-isopropylidene-6-O-(2-vinyloxyethyl)-D-galactopyranose.

Preferably, the side segment has a molar mass of greater than 1500 g/mol.

The reactive function Y is chosen, for example, from amine, hydrazine, hydrazone, azide, aldehyde (in particular masked aldehyde in the anomeric position of a saccharide ring), thiol, activated carboxylic acid, such as with N-hydroxysuccinimide, nitrile, haloalkyl, hydroxyl, maleimide, epoxy and alkoxyamine groups.

Depending on the constraints in terms of polymerization and construction of the architecture of the polymer, the reactive functions may or may not be protected on the monomers B and/or D. When the monomer B is N-acryloxysuccinimide, this monomer polymerizes without it being necessary to protect the reactive function. When the monomer D is a sugar derivative, it is necessarily protected, as is explained in the examples.

The protecting groups of isopropylidene type are removed in acidic medium, which releases the hydroxyl functions of the saccharide ring, giving the side segment a hydrophilic nature. This also allows an equilibrium to be established between the cyclic form and the acyclic form of the sugar, the latter form giving rise to an aldehyde function at the anomeric position of the sugar.

In a first embodiment of the invention, the reactive function Y is different than the reactive function X. Various preferential modes are indicated below:

X is a carboxylic function activated with an N-hydroxysuccinimide (for example if B is N-acryloxysuccinimide) and Y is a protected aldehyde function (for example if D is 1,2:3,4-di-O-isopropylidene-6-O-(2-vinyloxyethyl)-D-galactopyranose), and in this case the side segment is functionalized at the end with an amine, hydrazine or alkoxyamine function, as is clearly described in the examples. The function resulting from attaching the side segment onto the skeleton is a very stable function of peptide or hydrazinopeptide type.

X is a carboxylic function activated with an N-hydroxysuccinimide (for example if B is N-acryloxysuccinimide) and Y is a haloalkyl function (for example if D is chloroethyl vinyl ether), and in this case the side segment is functionalized at the end with an amine, hydrazine or alkoxyamine function. The function resulting from attaching the side segment onto the skeleton is a very stable function of peptide or hydrazinopeptide type.

X is an amine function (for example if B is 2-aminoethyl acrylate) and Y is a haloalkyl function (for example if D is chloroethyl vinyl ether), and in this case the side segment is functionalized at the end with an aldehyde function. The function resulting from attaching the side segment onto the skeleton is a function of imine type stabilized by reduction to a secondary amine (for example by using NaBH₄).

X is an amine function (for example if B is 2-aminoethyl acrylate) and Y is a protected aldehyde function (for example if D is 1,2:3,4-di-O-isopropylidene-6-O-(2-vinyloxyethyl)-D-galactopyranose), and in this case the side segment is functionalized at the end with an aldehyde function. The function resulting from attaching the side segment to the skeleton is a function of imine type stabilized by reduction to a secondary amine (for example using NaBH₄).

X is an amine function (for example if B is 2-aminoethyl acrylate) and Y is a protected aldehyde function (for example if D is 1,2:3,4-di-O-isopropylidene-6-O-acryloyl-D-galactopyranose), and in this case the side segment is functionalized at the end with a carboxylic function (introduced during the synthesis of said side segment by the RAFT technique). The end carboxylic function is activated (for example using dicyclohexylcarbodiimide, DCC) in order to covalently bond said side segment to one of the functions X of the skeleton. The function resulting from attaching the side segment to the skeleton is a very stable function of peptide type.

More preferably, X is an amine function (for example if B is 2-aminoethyl acrylate) and Y is a carboxylic function activated with an N-hydroxysuccinimide (for example if D is N-acryloxysuccinimide), and in this case the side segment is functionalized at the end with a carboxylic function (introduced during the synthesis of the side segment via the RAFT technique). In this case, it is necessary first to covalently couple the biological ligands to the functions Y of the side segment. After the residual functions Y have been blocked (for example with aminoethylmorpholine), the terminal carboxylic function of the side segment is activated (for example using dicyclohexylcarbodiimide, DCC) in order to covalently bond said side segment to one of the functions X of the skeleton. The function resulting from attaching the side segment to the skeleton is a very stable function of peptide type.

In a second embodiment of the invention, the reactive function Y is identical to the reactive function X.

In this case, it is preferable for the reactive functions X and Y to be functions that are protected and deprotected under predetermined conditions. In particular, it is necessary to avoid reactions leading to intra-segment crosslinking phenomena, which would be prejudicial to the oriented three-dimensional structure of the branched polymer according to the invention. Preferably, X and Y are carboxylic functions activated with an N-hydroxysuccinimide (for example if B and D are N-acryloxysuccinimide), and in this case the side segment is functionalized at the end with a thiol function (introduced during the synthesis of the side segment via the RAFT technique). In this case, it is necessary first to covalently couple the biological ligands to the functions Y of the side segment. After the residual functions Y have been blocked (for example with aminoethylmorpholine), the terminal thiol function of the side segment is converted into an amine function (for example using N-iodoethyltrifluoroacetamide) in order to covalently bond said side segment onto one of the functions X of the skeleton. The function resulting from attaching the side segment to the skeleton is a very stable function of peptide type.

Preferably, X and Y are functions of protected aldehyde type (for example if B is 1,2:3,4-di-O-isopropylidene-6-O-acryloyl-D-galactopyranose and if D is 1,2:3,4-di-O-isopropylidene-6-O-(2-vinyloxyethyl)-D-galactopyranose), and in this case the side segment is functionalized at the end with an amine or hydrazine function. In this case, it is necessary first to deprotect the sugars of the skeleton in order to covalently bond the side segment onto one of the deprotected functions X of the skeleton. The function resulting from attaching the side segment to the skeleton is a function of imine type stabilized by reduction to a secondary amine (for example using NaBH₄). After the residual functions X on the skeleton have been blocked (for example with ethanolamine or, preferably, with aminoethylmorpholine), the sugars of the side segments are in turn deprotected in order to achieve the covalent coupling of biological ligands.

In another particular embodiment of the invention, the polymer skeleton also comprises a “spacer” segment intercalated between the first segment and the second segment, consisting of a linear homopolymer resulting from the polymerization of a hydrophilic monomer E, said monomer not bearing a reactive function.

The monomer E is chosen from acrylamide, methacrylamide and N-vinylpyrrolidone derivatives. Preferably, the monomer E is N-acryloylmorpholine (NAM).

The monomer E may be identical to or different than the monomer C, preferably identical since the polymerization reaction is simpler.

The spacer segment has a molar mass of greater than 5000 g/mol, preferably 10,000 g/mol.

The spacer segment is obtained by living free-radical polymerization of the monomer E, for instance reversible termination polymerization (using nitroxide radicals), atom transfer polymerization (ATRP), and preferably reversible addition-fragmentation chain-transfer polymerization (RAFT).

This spacer segment is synthesized consecutively to the first segment or to the second segment, depending on the nature of the monomers and on the polymerization technique adopted.

The present invention also relates to a conjugate comprising at least one biological ligand fixed to a polymer.

When the polymer according to the invention comprises no side segments, the biological ligands are fixed directly or indirectly to the polymer via reactive functions X of the monomer B.

When the polymer according to the invention comprises at least one side segment, the biological ligands are fixed directly or indirectly to the side segments. In a first fixing embodiment, the biological ligands are fixed in a first step to the side segment via functions Y and the side segments-biological ligands assembly is then fixed to the polymer skeleton optionally comprising a spacer segment. In a second, preferential, fixing embodiment, the biological ligands are fixed, again via functions Y, to the side segments, said side segments having been fixed beforehand via their reactive end to the reactive functions X of the polymer skeleton.

The expression “indirect fixing” means fixing by means of a noncovalent interaction.

Any known means based, for example, on affinity phenomena, especially between biological molecules, for instance the biotin/streptavidin interaction, may be used for a noncovalent interaction. For example, biotin is introduced onto the polymer by covalent coupling onto the reactive functions X and the fixing of the biological ligand to the polymer is provided by the presence of streptavidin introduced by coupling to the biological ligand. In another example, streptavidin is introduced onto the polymer by covalent coupling to the reactive functions X and the fixing of the biological ligand to the polymer is provided by the presence of biotin introduced by coupling to the biological ligand.

The expression “direct fixing” means fixing by covalent coupling. Many methods for introducing reactive functions onto a biological ligand are available: for proteins, antigens, antibodies or polypeptides, see, for example “Chemistry of protein conjugation and crosslinking”, Wong S. S., CRC Press, Boca Raton, 1991 or “Bioconjugate techniques”, Hermanson G. T., Academic Press, San Diego, 1996. For nucleic acids, a polynucleotide is synthesized, for example, by a chemical method on a solid support containing a function that is reactive at any point in the chain such as, for example, the 5′ end or the 3′ end or on a base or on an internucleotide phosphate or on position 2′ of the sugar (see “Protocols for Oligonucleotides and Analogs, Synthesis and Properties” edited by S. Agrawal, Humana Press, Totowa, N.J.). Methods for introducing reactive functions onto haptens are given especially in “Preparation of antigenic steroid-protein conjugate”, F Kohen et al., in Steroid immunoassay, Proceedings of the fifth tenovus workshop, Cardiff, April 1974, ed. E H D Cameron, S H. Hillier, K. Griffiths, such as, for example, the introduction of a hemisuccinate function in position 6, 11, 20 or 21, a chloroformat function in position 11 or a carboxymethyl function in position 6, in the case of progesterone. It is not necessarily obligatory to specifically introduce a reactive function onto the ligand. For example, in the case of a biological ligand of protein type having a lysine-sufficient composition, the amines borne by the lysine side chain may be used for the coupling.

The biological ligand is coupled to the polymer by forming a covalent bond between the two complementary reactive functions, one borne by the biological ligand and the other by the polymer. For example, a primary amine function may be coupled to a carboxylic acid activated, for instance, with N-hydroxysuccinimide, or to an aldehyde, an alkoxyamine function with a ketone or an aldehyde, a hydrazine function with an aldehyde, or a thiol function with a haloalkyl or a maleimide. In the case of a coupling between an amine and an aldehyde, it is preferable to reduce the imine formed, either simultaneously by the action of NaBH₃CN, or in a subsequent step by the action of NaBH₄ or NaBH₃CN.

Another subject of the present invention is a device for capturing a target molecule with the aim of detecting it and/or assaying it and/or purifying it, comprising a solid support on which is immobilized a polymer-biological ligand conjugate.

The term “solid support” as used herein includes any material on which the conjugate may be immobilized for use in diagnostic tests, in affinity chromatography and in separation processes. Natural, synthetic, chemically modified or unmodified materials may be used as solid support, especially polymers such as polyvinyl chloride, polyethylene, polystyrenes, polyacrylate, polyamide, or copolymers based on vinylaromatic monomers, alkyl esters of α,β-unsaturated acids, unsaturated carboxylic acid esters, vinylidene chloride, dienes or compounds containing nitrile functions (acrylonitrile); polymers of vinylchloride and of propylene, and the polymer of vinylchloride and of vinyl acetate; copolymers based on styrenes or substituted styrene derivatives; synthetic fibers such as nylon; mineral materials such as silica, glass, ceramic or quartz; latices and magnetic particles; metallic derivatives. The solid support according to the invention may be, without limitation, in the form of a microtitration plate, a sheet, a cone, a tube, a well, beads, particles or the like, or a flat support, for instance a silica or silicon wafer. The material is either hydrophilic or hydrophobic intrinsically or following a chemical modification, such as, for example, a hydrophilic support made hydrophobic.

For example, the surface of a silica wafer is made hydrophobic by silanization, using an alkylsilane, for instance n-octadecylmethyldichlorosilane, n-octadecyldimethylchlorosilane or n-octadecyltrichlorosilane.

In one embodiment, the polymer-biological ligand conjugate is immobilized on the solid support by covalent bonding.

For example, if A is t-butyl acrylate, the skeleton is immobilized on a silica wafer silanized with an aminosilane, by a transamidation reaction between the t-butyl ester functions of the first segment of the skeleton and the surface amine functions of the support, or by a hydrolysis reaction of the t-butyl ester functions of said first segment followed by an activation of the resulting carboxylic functions (using dicyclohexylcarbodiimide), in order to produce a covalent bond of peptide type with the amine functions at the surface of the support.

In one preferred embodiment according to the invention, the polymer-biological ligand conjugate is immobilized on the solid support by adsorption using an interaction of hydrophobic-hydrophobic type between the first segment of the polymer and the surface of the support which is, in this case, hydrophobic.

To allow the detection and/or quantification and/or purification of the target molecule, the biological ligand is capable of forming a ligand/antiligand capture complex. In particular, said antiligand constitutes the target molecule. Depending on the nature of the target to be detected, a person skilled in the art will choose the nature of the biological ligand to be fixed to the polymer. By way of example, in order to demonstrate a target molecule of nucleic acid type, the biological ligand may be a nucleic acid with sufficient complementarity to the target to hybridize specifically depending on the reaction conditions and especially the temperature or the salinity of the reaction medium.

A step of detecting the target molecule may be necessary, as in the case of a sandwich hybridization (see, for example, WO 91/19862), or the target molecule may be directly labeled, such as after an enzymatic amplification technique of PCR (polymerase chain reaction) type which incorporates a fluorescent nucleotide (see DNA probes, 2nd edition, Keller G. H. and Manak M., Stockton Press, 1993).

During the various covalent coupling steps described previously, it is desirable to block the reactive functions X or Y that have not reacted, by the action of a small chemical molecule. For example, during the fixing of biological ligands bearing an amine function to a polymer comprising no side segment and comprising an NAS derivative as monomer B, the residual activated ester functions of the skeleton are blocked by reaction of a molecule of aminoalkane type, for instance ethylamine, propylamine, butylamine or hexylamine, or of amino alcohol type, for instance ethanolamine, 3-amino-1-propanol, 4-amino-1-butanol, 5-amino-1-pentanol, 6-amino-1-hexanol or, preferably, aminoethylmorpholine. The same molecule is used to block the residual aldehyde functions of the monomer D of the side segment after fixing the biological ligands to said side segment. Another advantage of aminoethylmorpholine is that it provides additional hydrophilicity to the conjugate.

In another example, during the fixing of biological ligands to a polymer comprising no side segment and comprising an aminoethyl acrylate derivative as monomer B, the residual amine functions of the skeleton are blocked by reaction of a molecule of acid anhydride type, for instance acetic anhydride, or of acid chloride type, for instance acetyl chloride.

A subject of the present invention is also a process for synthesizing a polymer according to the invention, in which the linear skeleton of the polymer is prepared by growing chains by one of the methods generally used for synthesizing block copolymers. Among these methods, the one preferably chosen is either sequential addition of the monomer(s) corresponding to one of the two segments and then of the monomer(s) corresponding to the other segment, or presynthesis of one of the two segments, said segment then being used as a macroprimer or macrotransfer agent for the synthesis of the other segment.

Any one of the living free-radical polymerization techniques described previously may be used for the synthesis of each segment, preferably reversible addition/fragmentation chain-transfer (RAFT) polymerization.

The first segment and the second segment are synthesized either by the same technique or by a combination of two different techniques.

A person skilled in the art will select a strategy for synthesizing the skeleton (order of introduction of the monomers) as a function of the nature of the monomers A, B and C chosen to make the skeleton and as a function of the polymerization technique selected.

For example, if A is t-butyl acrylate, if B is NAS and if C is NAM, the skeleton is prepared by the macrotransfer agent method using the RAFT polymerization technique. In a first stage, a random copolymer of NAS and of NAM is synthesized by the RAFT technique. In a second stage, this copolymer is used as a macrotransfer agent during the polymerization of t-butyl acrylate by the RAFT technique. This results in a lengthening of the copolymer chains with a homopoly (t-butyl acrylate) segment.

In another example, if A is styrene, if B is NAS and if C is NAM, the skeleton is prepared by the method of sequential addition of monomers using the RAFT polymerization technique. In this case, the styrene is polymerized giving rise to the first segment, and a mixture of NAS and NAM is then introduced in order to synthesize the second segment consecutively to the first. The reverse order may also be performed.

When the polymer skeleton comprises a spacer segment, the synthetic process is similar, either by sequential addition of the monomers corresponding to the various segments, or by successive synthesis of macroprimers, again by any of the living free-radical polymerization techniques.

In the case of the synthesis of a polymer comprising at least one side segment, this synthetic process comprises the following steps:

the linear skeleton of the polymer is prepared by growing chains starting from one of the ends of the polymer as described previously,

the side segment is prepared independently by means of a controlled polymerization technique chosen from the techniques comprising living cationic polymerization, living anionic polymerization and free radical reversible addition/fragmentation chain-transfer (RAFT) polymerization, and a reactive function capable of reacting with the reactive function X of the monomer B present on the skeleton is then introduced onto said side segment, at one end,

the linear skeleton and several side segments are placed in contact to allow the covalent bonding of the side segments along the skeleton.

By way of example, if A is t-butyl acrylate, if B is NAS and if C is NAM, the skeleton is prepared by the macrotransfer agent method using the RAFT polymerization technique. In a first stage, a random copolymer of NAS and NAM is synthesized by the RAFT technique. In a second stage, this copolymer is used as a macrotransfer agent during the polymerization of t-butyl acrylate by the RAFT technique. This results in a lengthening of the copolymer chains with a homopoly (t-butyl acrylate) segment.

As regards the side segment, if D is 1,2:3, 4-di-O-isopropylidene-6-O-(2-vinyloxyethyl)-D-galactopyranose, the side segment is obtained by living cationic polymerization. Given the presence of reactive functions X of activated ester type on the skeleton, the side segment is functionalized at the end with an amine or hydrazine function.

A large number of side segments are introduced into a solution of the skeleton in the presence of triethylamine. The function resulting from fixing the side segments to the skeleton is a very stable function of peptide or hydrazinopeptide type.

In another example, if A is t-butyl acrylate, if B is 2-aminoethyl acrylate and if C is NAM, the skeleton is prepared by the macrotransfer agent method using the RAFT polymerization technique. In a first stage, a random copolymer of B and C is synthesized by the RAFT technique. In a second stage, this copolymer is used as a macrotransfer agent during the polymerization of t-butyl acrylate by the RAFT technique. This results in a lengthening of the copolymer chains with a homopoly(t-butyl acrylate) segment.

As regards the side segment, if D is chloroethyl vinyl ether, the side segment is obtained by living cationic polymerization. Given the presence of reactive functions X of amine type on the skeleton, the side segment is functionalized at the end with an aldehyde function.

A large number of side segments are introduced into a solution of the polymer skeleton (said skeleton comprising the monomers A, B, C and optionally E) . The function resulting from attaching the side segment to the skeleton is a function of imine type stabilized by reduction to a secondary amine (for example using NaBH₄) .

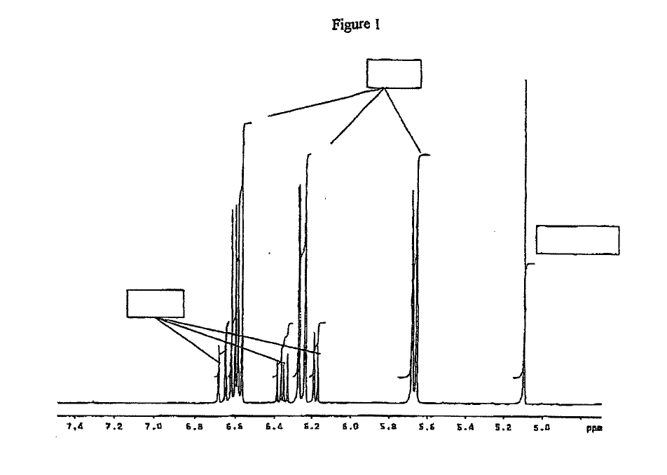

FIG. 1 represents an example of the ¹H NMR spectrum for the kinetic monitoring of the consumption of the monomers as described in Example 1. The trioxane peak is visible at 5.1 ppm (right-hand rectangle), the peaks corresponding to the NAM protons are indicated by the lines coming from the center rectangle, and the peaks corresponding to the NAS protons are indicated by the lines coming from the left-hand rectangle.

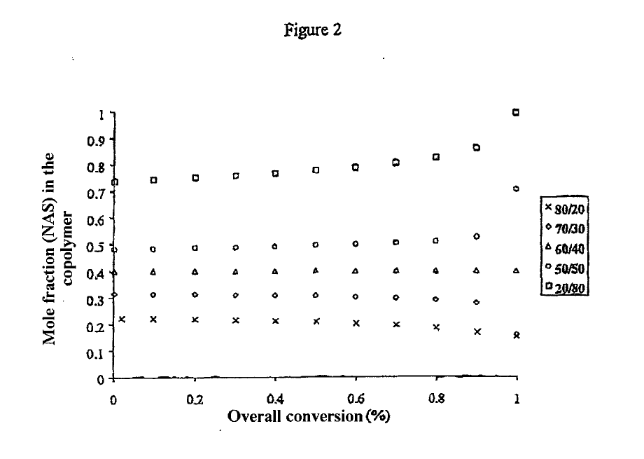

FIG. 2 shows the change in the composition of the NAM/NAS of copolymer as a function of the degree of conversion (%) for various molar ratios of the NAM/NAS monomers in the initial blend: 80/20; 70/30; 60/40; 50/50; 20/80 as described in Example 1.

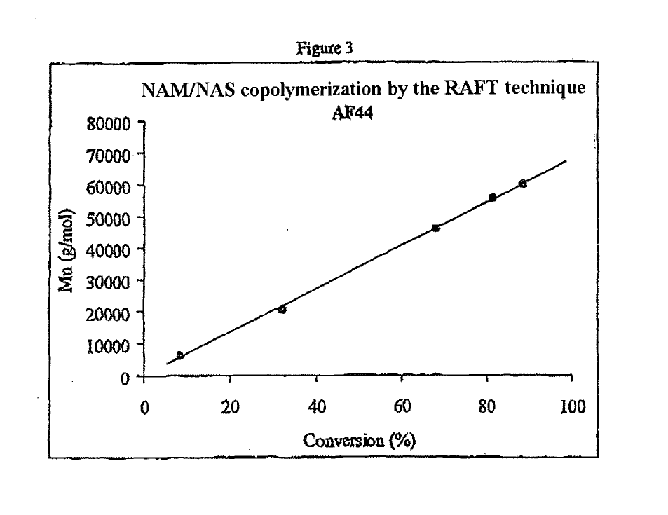

FIG. 3 shows the molar mass expressed in g/mol of the copolymer AF 44 as a function of the degree of conversion of the monomers expressed in % (see Example 2).

The examples that follow illustrate a number of advantages of the invention without, however, limiting the scope thereof.

### EXAMPLE 1

### Conventional Free-Radical Synthesis of NAM/NAS Skeletons

General Procedure:

The N-acryloylmorpholine (NAM, sold by Polysciences, Inc., reference 21192) is distilled before use in polymerization.

The N-acryloxysuccinimide (NAS, sold by Acros, reference 40030) is purified by chromatography on a column of silica before use in polymerization.

The dioxane (solvent) (sold by SDS, reference 27.053-9) is distilled over LiAlH₄ before use.

The azobisisobutyronitrile AIBN (polymerization initiator) (Fluka, reference 11630) is recrystallized from ethanol.

The trioxane (internal reference for the ¹H NMR monitoring, sold by Janssen-Chimica, reference 14.029.61) is used as supplied.

The experiments for the homopolymerization of NAM and for the copolymerization of NAM with NAS were carried out in a 100 mL three-necked round-bottomed flask equipped with a magnetic stirring system and a nitrogen inlet.

The monomers, the trioxane and the solvent are introduced into the flask and the mixture is degassed for one hour by sparging with nitrogen in order to remove all trace of dissolved oxygen.

The reaction mixture is maintained at 60° C. for 15 minutes.

During this quarter of an hour, the AIBN, dissolved in 0.5 mL of dioxane, is degassed by sparging with nitrogen.

It is then rapidly introduced into the flask using a syringe placed beforehand under a stream of nitrogen.

This is time zero for the polymerizations.

Throughout the manipulation, the nitrogen inlet is maintained over the reaction medium, so as to prevent any preferential sites of polymerization (syringe).

Samples of about 500 μL are taken at given times, transferred into flasks containing traces of hydroquinone (sold by Janssen-Chimica, reference 123-3169) and placed in an ice bath.

At the end of the reaction, the polymer is recovered by precipitation from ether and then dried under vane-pump vacuum overnight.

Kinetic Monitoring of the Polymerization:

The kinetic monitoring of the consumption of the monomers is performed by ¹H NMR (Nuclear Magnetic Resonance) on a Varian Unity Plus 500 MHz spectrometer.

The samples to be analyzed are prepared by mixing 300 μL of each withdrawn sample with 300 μL of deuterated solvent, CDCl₃. The ¹H NMR analysis is performed by irradiating the dioxane peak. This method has the advantage of analyzing the reaction medium without evaporating the synthesis solvent and thus avoids possible transformations of the products.

The reduction in the peaks relating to the vinyl protons of the monomers is monitored as a function of time relative to an internal reference, trioxane. Trioxane has the particular feature of having a ¹H NMR peak in the form of a strong, sharp singlet that is isolated from the vinyl protons of the two monomers, NAM and NAS (see FIG. 1 for an example of the NMR spectrum).

The conversions of the two monomers are obtained by:

$${C}_{NAM} = 1 \frac{{( \frac{{H}_{NAM}}{{H}_{trioxane}} )}_{t}}{{( \frac{{H}_{NAM}}{{H}_{trioxane}} )}_{0}} \qquad and \qquad {C}_{NAS} = 1 \frac{{( \frac{{H}_{NAS}}{{H}_{trioxane}} )}_{t}}{{( \frac{{H}_{NAS}}{{H}_{trioxane}} )}_{0}}$$

with C_{NAS}: NAS conversion,

C_{NAM}: NAM conversion,

H_{NAM}: integral relative to one NAM proton,

H_{NAS}: integral relative to one NAS proton,

Hₜᵣᵢₒₓₐₙₑ: integral relative to the six trioxane protons

The kinetic study performed made it possible to determine the copolymerization reactivity rates for this pair of monomers: I_{NAS}=0.63 and I_{NAM}=0.75

These values indicate that the composition drift is very low during the copolymerization, which amounts to stating that the macromolecular chains formed are of very homogeneous composition, and have a composition similar to that of the initial monomer blend (FIG. 2, which shows the change in the composition of the NAM/NAS copolymer as a function of the degree of conversion (%) for various molar ratios of the NAM/NAS monomers in the initial blend: 80/20; 70/30; 60/40; 50/50; 20,80).

This is particularly true for the “azeotropic” composition, which corresponds to an NAM/NAS ratio of 60/40.

The operating conditions used and the characteristics of the various copolymers synthesized are summarized in the tables below:

TABLE A
Copolymers of variable NAM/NAS molar ratio and of similar
molar masses of the order of 100,000 g/mol.
Initial concentration of monomers in the tests:

| Copolymer | [NAM]₀ | [NAS]₀ | [Trioxane] |
|---|---|---|---|
| reference | (mol · L⁻¹) | (mol · L⁻¹) | (mol · L⁻¹) |
| COPO 1 | 0.810 | 0.200 | 0.063 |
| COPO 2 | 0.703 | 0.300 | 0.059 |
| COPO 3 | 0.590 | 0.400 | 0.050 |
| COPO 4 | 0.500 | 0.500 | 0.040 |

All these tests are performed in dioxane at 60° C., in the presence of a concentration of initiator [AIBN]=0.005 mol.L⁻¹.

Characteristics of the Copolymers:

| | Conversion | | Composition | | | |
|---|---|---|---|---|---|---|
| Copolymer | (%) | | NAM/NAS | Mₙ** | Mₚₑₐₖ | |
| reference | NAM | NAS | (*) | (g · mol⁻¹) | (g · mol⁻¹) | Iₚ** |
| COPO 1 | 94.6 | 98.3 | 80/20 | 79 200 | 145 500 | 2.0 |
| COPO 2 | 89.0 | 91.7 | 70/30 | 94 000 | 153 500 | 1.8 |
| COPO 3 | 82.5 | 81.7 | 60/40 | 94 000 | 137 800 | 1.7 |
| COPO 4 | 82.0 | 76.6 | 51/49 | 101 200 | 162 000 | 1.8 |

(*) obtained from the copolymerization equation using the degrees of reactivity determined previously.
**Mₙ is the number-average molar mass of the polymer chains formed, Mₚₑₐₖ corresponds to the molar mass of the majority population, and Iₚis the polydispersity index reflecting the homogeneity of the masses of the polymer chains (the closer the value of Iₚto 1, the more the polymer chains are homogeneous in mass).

These data are obtained by steric exclusion chromatography (SEC) in DMF, with polystyrene calibration, i.e.:

Column: Polymer Laboratories Gel Mixed column, type C; pump: Waters 510; UV detector: Waters 484; differential refractometric detector: Waters 410; eluent: dimethylformamide (DMF); flow rate: 0.5 mL.min⁻1; temperature: 55° C.; standards: polystyrene standards.

The copolymer masses measured by the SEC/LSD reference technique are given below:

| Copolymer | Mₙ* | Mₚₑₐₖ | |
|---|---|---|---|
| reference | (g · mol⁻¹) | (g · mol⁻¹) | Iₚ* |
| COPO 1 | 98 200 | 137 000 | 1.8 |
| COPO 2 | 112 300 | 142 600 | 1.8 |
| COPO 3 | 121 200 | 131 900 | 2.0 |
| COPO 4 | 87 100 | 82 000 | 1.4 |

*Conditions of analysis by Steric Exclusion Chromatography (SEC) coupled to a dynamic light-scattering detector (LSD):

Columns: Ultra Hydrogel 500 and 2000 (Waters); pump: Waters 510; UV detector: Waters 484; differential refractometric detector: Waters 410; dynamic light-scattering detector: three angles, miniDawn, Wyatt Technology; eluent: 0.05 M pH 9.3 borate buffer; flow rate: 0.5 mL.min⁻¹.

TABLE B
NAM/NAS or acrylamide/NAS (AAm/NAS) copolymers
of constant molar ratio: 80/20 and of variable
molar masses Synthetic operating conditions

| Copoly- | | | | | Polymeri- | Comono- |
|---|---|---|---|---|---|---|
| mer | | | | | zation | mer used |
| refer- | [M] | [AIBN] | T | | time | with the |
| ence | (mol · L⁻¹) | (% [M]) | (° C.) | Solvent | (hours) | NAS |
| FD2 | 1.00 | 1 | 60 | DMF | 4 | AAm |
| FD3 | 0.80 | 1 | 60 | DMF | 4 | AAm |
| FD20 | 0.65 | 1 | 60 | DMF | 4 | AAm |
| FD5 | 1.00 | 0.5 | 60 | DMF | 2 | AAm |
| FD19 | 1.00 | 1 | 55 | DMF | 4 | AAm |
| FD21 | 1.00 | 1 | 50 | DMF | 4 | AAm |
| FD14 | 1.00 | 0.5 | 60 | DMF | 2 | NAM |
| FD15 | 1.00 | 0.5 | 60 | Toluene | 4 | NAM |
| FD16 | 1.00 | 0.5 | 60 | Dioxane | 2 | NAM |

[M]=[NAM]+[NAS] or [AAm]+[NAS] with [ ] to indicate the concentration.

Measurement of the Copolymer Sizes

| | SEC/LSD* | | |
|---|---|---|---|
| Copolymer | Mₙ | Mₚₑₐₖ | |
| reference | (g · mol⁻¹) | (g · mol⁻¹) | Iₚ |
| FD2 | 33 100 | 37 700 | 1.5 |
| FD3 | 27 300 | 25 500 | 1.7 |
| FD20 | 12 090 | — | 1.7 |
| FD5 | 45 200 | 55 500 | 1.4 |
| FD19 | 27 600 | 32 400 | 1.8 |
| FD21 | No polymerization | | |
| FD14 | 79 400 | 89 600 | 1.6 |
| FD15 | — | — | — |
| FD16 | 107 400 | 133 500 | 1.6 |

*Conditions of analyses by Steric Exclusion Chromatography (SEC) coupled to a dynamic light-scattering detector (LSD):

Columns: Ultra Hydrogel 500 and 2000 (Waters); pump: Waters 510; UV detector: Waters 484; differential refractometric detector: Waters 410; dynamic light-scattering detector: three angles, miniDawn, Wyatt Technology; eluent: 0.05 M pH 9.3 borate buffer; flow rate: 0.5 mL.min⁻¹.

Analysis by ¹³C NMR of the Copolymer of NAM and NAS: Counting and Assignment of the Carbons in the Analysis:

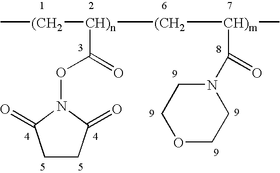

| | Chemical shift in ppm | |
|---|---|---|
| Position of the carbon | (DMSO-d₆, 360 K) | |
| 1 | 35 | (broad line) |
| 2 | 41 | (broad line) |
| 3 | 169-171 | (broad line) |
| 4 | 169 | (sharp line) |
| 5 | 25 | (sharp line) |
| 6 | 35 | (broad line) |
| 7 | 41 | (broad line) |
| 8 | 171-173 | (broad line) |
| 9 | 65 | (sharp line) |

### EXAMPLE 2

### Synthesis of NAM/NAS Skeletons by Controlled Free-Radical Polymerization of RAFT Type

The synthesis of NAM homopolymers and of random copolymers of NAM and NAS (80/20 molar ratio) was performed by the RAFT process, using thiobenzoylthioglycolic acid (I) (sold by Aldrich, reference 15,788-0) or, preferably, tert-butyl dithiobenzoate (II) as transfer agent of dithioester type.

This process makes it possible to obtain monodisperse chains and controllable molar masses as a function of the conversion.

The polymer chains formed bear at one of their ends a dithioester function (readily hydrolyzable to a thiol function, for example by the action of a primary amine), and at the other end either a carboxylic function or a t-butyl function using the dithioester I or II.

Synthesis of Tert-Butyl (or T-Butyl) Dithiobenzoate (II):

This synthesis was performed using a general method of reaction of thiol or of thiolate with a dithioester, described in the following article: Leon N. H., Asquith R. S., Tetrahedron, 26, 1719-1725 (1970).

150 ml of a solution of thiobenzoylthioglycolic acid at 0.016 mol.L⁻¹ in diethyl ether are added with vigorous stirring and at room temperature to 100 mL of an aqueous basic solution (0.1 N NaOH) of sodium t-butyl thiolate (sold by Aldrich, reference 35,930-0) at 0.028 mol.L⁻¹ (1.2 equivalents), in a 500 mL round-bottomed flask.

After reaction for 12 hours, the ether phase is washed with twice 500 mL of a basic aqueous solution (1 N NaOH) and then with 500 mL of aqueous 10% NaCl solution.

The t-butyl dithiobenzoate is purified by chromatography on silica gel (Kieselgel-60; CH₂Cl₃ eluent); the purified product is obtained in a yield of greater than 90%.

Procedure for the (Co)Polymerizations:

The various reagents are introduced into a reactor of Schlenk type at room temperature and the mixture is degassed by a sequence of freezing/vacuum/thawing cycles, and then placed under nitrogen.

The reaction mixture is brought to 60° C. and left stirring for about thirty hours. The polymer is precipitated from ether and dried under vane-pump vacuum.

Operating conditions of the homopolymerization of NAM (AF06 and AF37) and for the copolymerization of NAM with NAS (AF09, FD73 and AF44) by the RAFT process:

| Test | | | [Monomers] | | [Monomers]/ | (Dithioester]/ |
|---|---|---|---|---|---|---|
| reference | Dithioester | NAM/MAS | (mol · L⁻¹) | Solvent | [dithioester] | [AIBN] |
| AF06 | I | 100/0 | 3.9 | dioxane | 350 | 3.3 |
| AF37 | II | 100/0 | 3.9 | dioxane | 350 | 3.3 |
| AF09 | I | 80/20 | 3.9 | dioxane | 350 | 3.3 |
| FD73 | I | 80/20 | 4 | dioxane | 350 | 4 |
| AF44 | II | 80/20 | 3.9 | dioxane | 350 | 3.3 |

[X] means concentration of reagents X.

Characteristics of the polymers obtained (SEC coupled to an LSD detector):

| | Reaction | Monomer | | | |
|---|---|---|---|---|---|
| Test | time | conversion | Mₙ | Mₚₑₐₖ | |
| reference | (hours) | (%) | (g · mol⁻¹) | (g · mol⁻¹) | Iₚ |
| AF06 | 2 | 5 | 33000 | 41000 | 1.18 |
| | 4 | 11 | 39000 | 48000 | 1.17 |
| | 6 | 26 | 49000 | 52000 | 1.11 |
| | 8 | 39 | 55000 | 61000 | 1.11 |
| | 30 | 68 | 75000 | 81000 | 1.13 |
| AF37 | 1.25 | 9 | 9900 | 9000 | 1.17 |
| | 1.66 | 32 | 22700 | 24000 | 1.02 |
| | 2 | 42 | 29700 | 30800 | 1.02 |
| | 3.5 | 68 | 53700 | 53600 | 1.03 |
| | 5.5 | 78 | 59700 | 60200 | 1.05 |
| | 7 | 86 | 68200 | 67600 | 1.06 |
| | 22 | 97 | 76100 | 78800 | 1.12 |
| AF09 | 2 | 7 | 41300 | 46700 | 1.25 |
| | 4 | 13 | 47400 | 51900 | 1.29 |
| | 6 | 19 | 54000 | 57000 | 1.23 |
| | 8 | 29 | 57600 | 62000 | 1.24 |
| | 24 | 60 | 80000 | 88500 | 1.26 |
| | 33 | 74 | 80000 | 92600 | 1.28 |
| FD73 | 30 | 100 | 95000 | 103000 | 1.30 |
| AF44 | 2.75 | 8 | 6400 | 5900 | 1.18 |
| | 3.5 | 32 | 20700 | 20300 | 1.02 |
| | 6 | 68 | 46000 | 43700 | 1.03 |
| | 8 | 82 | 55700 | 52300 | 1.04 |
| | 10 | 89 | 60100 | 56700 | 1.05 |
| | 32 | 98 | 69900 | 64100 | 1.09 |

The SEC/LSD conditions are described for Table B of Example 1.

Depending on the reaction kinetics, it is possible to control the molar mass Mₚₑₐₖ of the polymer example, to obtain a molar mass of greater than 40,000 g/mol for the majority population.

The molar masses of the synthesized polymers increase as the conversion increases, in a perfectly linear manner (FIG. 3, which shows the molar mass expressed in g/mol of the copolymer AF44 as a function of the degree of conversion expressed in %), which makes it possible to envision the synthesis of copolymers of variable length depending on the conversion at which the copolymerization is stopped, and in a totally controlled and reproducible manner. Furthermore, the polydispersity indices, Iₚ are very low, particularly when the dithioester II is used, which indicates that the polymer chains formed are very homogeneous in size.

### EXAMPLE 3

### Synthesis of a Skeleton Containing tBuA-b-NAM/NAS Blocks by the RAFT Polymerization Technique

The RAFT process allows the synthesis of a block skeleton, containing a hydrophobic block of poly(tert-butyl acrylate, tBuA), and a hydrophilic and functional block consisting of an NAM/NAS random copolymer.

In fact, this diblock copolymer is obtained in two steps: one of the two blocks is synthesized in a first stage, and these polymer chains (bearing a dithioester function at one of their ends) are then used as (macro)transfer agent during the polymerization of the monomer corresponding to the second block. Diblock copolymers are thus obtained, as a mixture with a small amount of homopolymer of the second block.

The copolymer NAM/NAS FD73 was used as (macro)transfer agent during the polymerization of t-butyl acrylate.

Procedure:

Copolymer FD73 (2.5 g) of Example 2, tBuA (2.9 g, i.e. the amount required to lengthen the FD73 copolymer chains by one block of 65,000 g.mol⁻¹ at 100% conversion, product supplied by Aldrich, reference 37,718-2), initiator AIBN (Fluka, reference 11630) ((macro)transfer agent/AIBN molar ratio of 4) are dissolved in 7.5 ml of dioxane in a reactor of Schlenck type.

The mixture is degassed by a sequence of freezing/vacuum/thawing cycles and is then placed under nitrogen. It is then brought to 60° C. and left stirring for 22 hours (66% conversion). After dilution with dichloromethane, the polymer is precipitated from ether, recovered by centrifugation and dried under vane-pump vacuum. The precipitate is only partially soluble in a borate buffer.

¹H NMR analysis of the insoluble fraction confirms the presence of poly(tBuA) and poly(NAM/NAS) units; this fraction thus corresponds to the copolymer containing tBuA-b-NAM/NAS blocks referenced FD77.

Given the estimation of the proportion of chains of FD73 that have effectively undergone the elongation and the conversion of the tBuA monomer, the length of the PtBuA block is about 250,000 g.mol⁻¹.

### EXAMPLE 4

### Synthesis of the Hydrophobic Segment of the Skeleton by Controlled Free-Radical Polymerization of RAFT Type

The synthesis of hydrophobic homopolymers of tert-butyl acrylate (tBuA), of tert-butylacrylamide (tBuAAm, sold by Aldrich, reference 41,177-9) and of octadecylacrylamide (ODAAm, sold by Polysciences Inc., reference 04673-10) was performed by the RAFT process, using thiobenzoylthioglycolic acid (I) sold by Aldrich, reference 15,788-0) or, preferably, t-butyl dithiobenzoate (II) (see Example 2) as transfer agent of dithioester type.

As explained previously, this process makes it possible to obtain monodisperse chains and controllable molar masses as a function of the conversion.

The polymer chains formed bear at one of their ends a dithioester function (which will allow the synthesis of a second hydrophilic block, consecutively to the first hydrophobic block), and at the other end either a carboxylic function or a t-butyl function, depending on whether the dithioester I or II, respectively, has been used as transfer agent.

It should be noted that the monomers tBuAAm and ODAAm are monosubstituted acrylamides, thus having a hydrogen on the amide function, which makes their polymerization difficult to control by another controlled free-radical polymerization process, for example the ATRP process.

Procedure for the Polymerizations:

The various reagents are introduced into a reactor of Schlenk type at room temperature, and the mixture is degassed by a sequence of freezing/vacuum/thawing cycles and then placed under nitrogen.

The reaction mixture is brought to 60° C. or 90° C. and left stirring for about 30 hours. In the case of tBuA, the polymer is purified by coevaporation of the residual monomer, the dioxane and the trioxane with acetonitrile (2×200 ml) and then dried under vane-pump vacuum. In the case of tBuAAm, the polymer is purified by coevaporation of the residual monomer, the dioxane and the trioxane with DMF (2×200 ml) and then dried under vacuum.

In the case of ODAAm, the polymer is purified by precipitation from ether and then dried under vane-pump vacuum.

Operating Conditions for the Homopolymerization of tBuA (MTC 901, AF41, AF49, AF60, AF72), tBuAAm (BDL1) and ODAAm (BDL2) by the RAFT process:

| | | Nature | | | | | |
|---|---|---|---|---|---|---|---|
| Polymer | | of the | [Monomer] | | [Monomer]/ | [Dithioester]/ | Temper- |
| reference | Dithioester | monomer | (mol · L⁻¹) | Solvent | [dithioester] | [AIBN] | ature |
| MTC 901 | I | tBuA | 3.4 | dioxane | 153 | 4.1 | 60° C. |
| AF41 | II | tBuA | 3.4 | dioxane | 350 | 3.3 | 60° C. |
| AF49 | II | tBuA | 4 | dioxane | 350 | 3.3 | 90° C. |
| AF60 | II | tBuA | 1 | dioxane | 350 | 3.3 | 90° C. |
| AF72 | II | tBuA | 4 | dioxane | 630 | 3.3 | 90° C. |
| BDL1 | II | tBuAAm | 1 | dioxane | 400 | 3.3 | 90° C. |
| BDL2 | II | ODAAm | 1 | dioxane | 154 | 3.3 | 90° C. |

[X] means: concentration of reagent X.

Characteristics of the Polymers (Molar Masses Obtained by SEC in DMF or THF*):

| | Reaction | Monomer | | | |
|---|---|---|---|---|---|
| Test | time | conversion | Mₙ | Mₚₑₐₖ | |
| reference | (hours) | (%) | (g · mol⁻¹) | (g · mol⁻¹) | Iₚ |
| MTC901 | 5.5 | 6 | 6500 | 11900 | 1.8 |
| | 7.0 | 8 | 6600 | 11900 | 1.8 |
| | 20.3 | 20 | 8900 | 14400 | 1.7 |
| | 29.0 | 30 | 11100 | 21100 | 1.7 |
| AF41 | 24 | 33 | 27600 | 39000 | 1.26 |
| AF49 | 0.33 | 34 | 16600 | 21900 | 1.27 |
| | 0.50 | 48 | 22800 | 31100 | 1.29 |
| | 0.67 | 58 | 25500 | 38300 | 1.38 |
| | 0.83 | 64 | 32500 | 43700 | 1.25 |
| | 1 | 69 | 33700 | 45800 | 1.29 |
| AF60 | 1 | 30 | 39900 | 46200 | 1.2 |
| | 4 | 64 | 46800 | 50300 | 1.3 |
| AF72 | 0.67 | 70 | 43000 | 58000 | 1.3 |
| BDL1* | 0.17 | 29 | ** | 4300 | ** |
| BDL1* | 0.42 | 35 | ** | 13800 | ** |
| | 1 | 59 | ** | 21100 | ** |
| | 1.5 | 65 | ** | 31000 | ** |
| | 2 | 69 | ** | 33400 | ** |
| BDL2* | 0.5 | 4 | 5400 | 5200 | 1.14 |
| | 1 | 46 | 12900 | 16400 | 1.28 |
| | 1.5 | 62 | 17900 | 23800 | 1.35 |
| | 18 | 74 | 17800 | 24900 | 1.36 |
| | 25 | 77 | 17600 | 25300 | 1.37 |

*The SEC/DMF conditions are described in Example 1, and the SEC/THF conditions are described in Example 8.
** Values not determinable due to the superposition of the peak for the residual monomer.

The molar masses of the synthesized polymers increase linearly as the conversion increases, which makes it possible to envision the synthesis of hydrophobic segments of variable length according to the conversion at which the polymerization is stopped, and to do so in a fully controlled and reproducible manner.

Furthermore, the polydispersity indices, Iₚ are low, particularly when the dithioester II is used, which indicates that the polymer chains formed are homogeneous in size.

Finally, by controlling the kinetics, it is possible to obtain for all the polymer combinations a molar mass M_{p eak} for the hydrophobic segment of greater than 10,000 g/mol.

### EXAMPLE 5

### Synthesis of a Triblock Skeleton tBuA-b-NAM-b-NAM/NAS Comprising an Intermediate Segment by Controlled Free-Radical Polymerization of RAFT Type

Poly(tBuA) AF72 (1 g, Mn=58,000 g.mol⁻¹, i.e. 1.72×10⁻⁵ mol) of Example 4, NAM (0.58 g) and initiator AIBN (1.5 mg) are dissolved in 2.5 ml of dioxane in a reactor of Schlenck type.

The mixture is degassed by a sequence of freezing/vacuum/thawing cycles and is then placed under nitrogen. It is then brought to 90° C. and left stirring.

After 40 minutes (72% conversion), a mixture of NAM and NAS dissolved in dioxane (0.362 g of NAM and 0.545 g of NAS in 4.6 ml of dioxane) is added (this mixture was degassed beforehand by three cycles of freezing/vacuum/thawing and then placed under nitrogen).

Given the residual amount of NAM at the time of the addition, the reaction mixture corresponds to an NAM/NAS molar ratio of 54/46. After 20 minutes, the reaction is stopped (81% conversion of the monomer blend).

The reaction mixture is precipitated in ether. The precipitate is recovered by filtration and dried under vane-pump vacuum. Separately, the residue obtained after concentrating the filtrate is dried under vacuum.

¹H NMR analysis of this residue confirms the presence of poly(tBuA) and poly(NAM/NAS) units; this fraction corresponds to the copolymer containing poly(tBuA-b-NAM-b-NAM/NAS) blocks, referenced AF73. Given the molar mass of the hydrophobic block (58,000 g.mol⁻¹) and also the conversion, the ¹H NMR analysis allows the length of the poly(NAM/NAS) segment to be estimated to be about 41,000 g.mol⁻¹ and that of the intermediate segment poly(NAM) to be 11,000 g.mol⁻.

### EXAMPLE 6

### Immobilization of the Skeleton Containing tBuA-b-NAM/NAS Blocks by Adsorption onto a Hydrophobic Mineral Flat Support

Production of a Hydrophobic Silicon Wafer by Silanization:

Plates of silica on silicon are cleaned in sulfochromic mixture at 120° C. for four hours, so as to regenerate the surface silanol functions.

After rinsing with MilliQ water, the contact angle of the water on these plates is less than 10° (not measurable). The plates are then dried under a stream of nitrogen and immediately immersed in a solution of n-octyldecylmethyldichlorosilane (ABCR reference S10 6625-0) at 2% (v/v) in toluene for two hours. After thorough rinsing with acetone, the plates are then dried under a stream of nitrogen, and then under air at 120° C. for two hours.

After this silanization step, the supports have a contact angle of water of 102° to 108°.

Adsorption of the Copolymer Containing tBuA-bNAM/NAS Blocks onto the Hydrophobic Wafer:

In order to confirm the influence of the presence of the tBuA hydrophobic block, two copolymers are compared:

the copolymer FD73 which does not comprise any tBuA blocks,

the copolymer FD77, which is a copolymer containing tBuA-b-NAM/NAS blocks.

The adsorption tests are performed by successive evaporation of drops of a solution of each of the two copolymers in chloroform, placed on the surface of the wafer. The experimental conditions are as follows:

application and successive evaporation of 5 drops of 45 μL (containing 200 μg.mL⁻¹ of copolymer in chloroform) on five supports,

one of the five supports is not rinsed,

rinsing of two of the five supports with chloroform (2 to 3 mL dropwise),

rinsing of the other two supports by immersion in borate buffer (pH=9.3, 20 minutes, 37° C.) and then in PBS Tween buffer (overnight at room temperature).

The contact angle of water is measured on each type of support. The results are given in the table below (comment: the immersion of a plate not comprising any copolymer in PBS Tween leads to a contact angle with water of 85°).

| | | Contact angle in |
|---|---|---|
| Copolymer | Rinsing | degrees |
| FD73 | None | 46 |
| | CHCl₃ | 101 |
| | Borate/PBS Tween | 69 |
| FD77 | None | not |
| | | measurable |
| | CHCl₃ | 101 |
| | Borate/PBS Tween | 40 |

The absence of a hydrophobic block in the copolymer FD73 does not allow it to remain adsorbed onto the hydrophobic support. It is removed during the washing in aqueous buffer.

The contact angle with water, which fell to 46° during the adsorption of the polymer, regains a value of 69° (which is close to that of the control, 85°) after the washes, which means that the polymer FD73 has been entrained by washing.

On the other hand, in the case of the block copolymer FD77, the contact angle is 40° after the washes, which means firstly that the nature of the surface has become highly hydrophilic, and secondly that the copolymer is hydrophobically adsorbed onto the support by means of its tBuA block since the washing does not entrain the polymer.

In both cases, washing with an organic solvent, CHCl₃, allows all of the copolymer to be entrained, whether or not it bears a block, which confirms in the case of FD77 that this copolymer was indeed immobilized by hydrophobic adsorption and not by covalent coupling, for example via a number of NAS functions of the skeleton which would have reacted with residual silanol functions at the surface of the support.

### EXAMPLE 7

### Synthesis of the Saccharide Monomer GVE

The monomer 1,2:3,4-di-O-isopropylidene-6-O-(2-vinyloxyethyl)-D-galactopyranose (known as GVE) is obtained from galactose via a Williamson reaction. After in situ formation of the alkoxide of the protected galactose, said product reacts by displacing the Cl (leaving group) of chloroethyl vinyl ether.

Reaction scheme for synthesis of the saccharide monomer GVE:

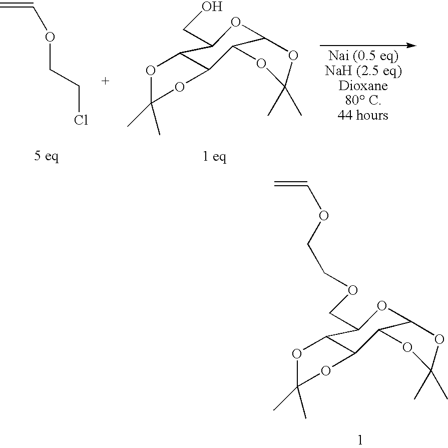

A solution of 1,2:3,4-di-O-isopropylidene-D-galactopyranose (10 g, 0.038 mol, Aldrich, reference D12,630-6) in dioxane (45 mL) is added dropwise to a suspension of NaH (4 g, 0.170 mol, Aldrich, reference 19,923-0) in dioxane (80 mL).

The temperature is raised to 80° C. Three hours later, NaI (2.9 g, 0.019 mol, Aldrich, reference 21,763-8) and a solution of 2-chloroethyl vinyl ether (20 g, 0.188 mol, Interchim >97%, reference C0174) in dioxane (45 mL) are added and the reaction mixture is left at 80° C. for two days.

The mixture is then diluted with ether, washed three times with saturated aqueous NH₄Cl solution and then with distilled water.

The organic phase is dried over MgSO₄, concentrated and then dried under vane-pump vacuum.

The crude product obtained is purified by chromatography on silica gel (eluent: ethyl acetate/pentane 20/80 V/V).

The desired product is obtained in the form of a yellow oil (60% yield).

Scheme 1

Saccharide Monomer GVE: Numbering and Assignment of the Carbons in the ¹³C NMR Analysis:

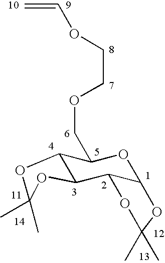

| | | Chemical shift in ppm |
|---|---|---|
| Position of the carbon | | (CDCl₃) |
| 1 | | 96.3 |
| 2 | | |
| 3 | | |
| 4 | | |
| 5 | {close oversize brace} | 66.5-71.1 |
| 6 | | |
| 7 | | |
| 8 | | |
| 9 | | 151.8 |
| 11 | | 108.5 |
| 12 | | 109.2 |
| 13 | | |
| | {close oversize brace} | 24.4-26.0 |
| 14 | | |

### EXAMPLE 8

### Synthesis of the PolyGVE Saccharide Grafts and of the Chloroalkyl PolyCEVE Grafts

Procedure:

The monomer 1,2:3,4-di-O-isopropylidene-6-O-vinyloxyethyl)-D-galactopyranose (GVE) is dried twice over CaH₂ (Aldrich, reference 21,332-2) before any living cationic polymerization experiment.

The polymerizations are performed in a reactor of Schlenk type. Each reagent is transferred therein via a cannula under nitrogen.

First, 20 ml of toluene (Merck, reference 1.08325.1000) (polymerization solvent) are introduced into the reactor and cooled to −20° C. (or −30° C.).

Acetaldehyde diethyl acetyl (Aldrich, reference A90-2) (dissolved in toluene) is added, followed by trimethylsilyl iodide (Aldrich, reference 19,552-9) (dissolved in toluene, 1.1 equivalents relative to the acetyl).

The solution is stirred for 30 minutes. The monomer (dissolved in toluene) is added, along with 0.2 equivalent of ZnCl₂ (Aldrich, reference 42,943-0) (dissolved in ether).

The reaction mixture is stirred at −20° C. (or −30° C.) under N₂, until an orange coloration appears, which is the sign of total conversion of the monomer.

In order to obtain polymer chain units ending with an aldehyde function, the reaction mixture is transferred into an aqueous KOH solution (pH=10-12) with vigorous stirring and the pH is rapidly adjusted to neutralization.

The heterogeneous mixture is left stirring for one hour and the organic phase is extracted with dichloromethane and washed several times with aqueous sodium thiosulfate solution and then with water.

The organic phase is dried over MgSO₄ and concentrated and the polymer is dried under vane-pump vacuum to constant mass.

Scheme 2

Numbering and Assignment of the Carbons in the ¹³C NMR Analysis of the GVE Homopolymer:

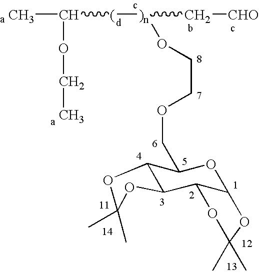

| | | Chemical shift in ppm |
|---|---|---|
| Position of the carbon | | (CDCl₃) |
| 1 | | 96.2 |
| 2 | | |
| 3 | {close oversize brace} | 69.8-71.0 |
| 4 | | |
| 5 | | 66.7 |
| 6 | | |
| 7 | {close oversize brace} | 68.0 |
| 8 | | |
| 11 | | |
| | {close oversize brace} | 108.0-109.5 |
| 12 | | |
| 13 | | |
| | {close oversize brace} | 24.4-26.0 |
| 14 | | |
| a | | 18.0-22.0 |
| b | | 48.0 |
| c | | 202.0 |
| d | | 39.0-41.0 |
| e | | 73.9 |

Tables Summarizing the PolyGVE Saccharide Grafts Synthesized

The first table shows the operating conditions of the tests and especially the amounts of reagents used.

| Test | [Diethyl | | | | |
|---|---|---|---|---|---|
| refer- | acetyl] | [TMSiI] | [GVE] | [ZnCl₂] | T |
| ence | (mol · L⁻¹) | (mol · L⁻¹) | (mol · L⁻¹) | (mol · L⁻¹) | (° C.) |
| FD30 | 0.010 | 0.012 | 0.078 | 0.0020 | −20 |
| FD84 | 0.030 | 0.036 | 0.350 | 0.0060 | −20 |
| FD33 | 0.005 | 0.006 | 0.075 | 0.0010 | −20 |
| FD96 | 0.030 | 0.034 | 0.68 | 0.0060 | −30 |
| FD67 | 0.005 | 0.006 | 0.152 | 0.0010 | −20 |
| FD100 | 0 | 0.008 | 0.094 | 0.0004 | −30 |

The characteristics of the homopolymers obtained are given in the table below:

| | Mₐ | Mₐ | Mₐ | Mₚₑₐₖ | |
|---|---|---|---|---|---|
| Test | (g · mol⁻¹) | (g · mol⁻¹) | (g · mol⁻¹) | (g · mol⁻¹) | Iₚ |
| reference | calculated | (¹H NMR) | (SEC) | (SEC) | (SEC) |
| FD30 | 2500 | 2950 | 1200 | 1700 | 1.26 |
| FD84 | 3800 | 3900 | 2100 | 2400 | 1.17 |
| FD33 | 5000 | 5800 | 900 | 1500 | 1.39 |
| FD96 | 7500 | 6200 | 3400 | 3900 | 1.11 |
| FD67 | 10,000 | 5800 | 3200 | 4500 | 1.24 |
| FD100 | — | 13,600 | 5500 | 6900 | 1.37 |

Table summarizing other types of grafts synthesized (polychloroethyl vinyl ether) polyCEVE:

The side segments prepared based on polyCEVE contain chloroalkyl functions which may be used for the covalent coupling of biological ligands.

This polymer was synthesized according to a procedure described in the article: Hëroguez V., Deffieux A. and Fontanille M., Makromol. Chem., Macromol. Symp., 32, 199 (1990).

| | | Mₐ | Mₐ | Mₚₑₐₖ | |
|---|---|---|---|---|---|
| | Mₐ (g · mol⁻¹) | (g · mol⁻¹) | (g · mol⁻¹) | (g · mol⁻¹) | Iₚ |
| REF | calculated | (¹H NMR) | (SEC) | (SEC) | (SEC) |
| FD21 | 5000 | 4200 | 4300 | 5400 | 1.20 |
| FD23 | 5000 | 5100 | 4400 | 5300 | 1.18 |
| FD29 | 2000 | 1900 | 1400 | 1900 | 1.33 |
| SC1F2 | 11,200 | * | 9300 | 9500 | 1.03 |
| SC4 | 30,000 | * | 22,000 | 27,700 | 1.13 |
| SC5C2 | 40,000 | * | 24,200 | 31,000 | 1.24 |

* Determination method not suitable for polymers with a molar mass of greater than 5000 g · mol⁻¹.

Analysis conditions by SEC in THF with polystyrene calibration:

Column: Polymer Laboratories Gel Mixed column, Type C,

Pump: Waters 510 High Performance Liquid Chromatography,

UV detector: Waters 2484,

Differential refractometric detector: Waters 410,

Eluent: tetrahydrofuran (THF),

Flow rate: 0.5 mL.min⁻¹,

Standards: polystyrene standards.

### EXAMPLE 9

### Deprotection of the PolyGVE Saccharide Grafts

Procedure:

The deprotection of the saccharide units of the polyGVE grafts is performed according to a procedure described in the literature.

polyGVE (1 g) is dissolved in 10 mL of a trifluoroacetic acid/water mixture (5/1, V/V).

The reaction medium is left at room temperature for one hour and then neutralized with saturated NaHCO₃ solution.

The mixture is then dialyzed (Spectra/Por 6 cellulose membrane, cut-off: 2000 g.mol⁻¹) to remove the salts, the residual trifluoroacetic acid and the acetone released during the deprotection. The dialyzed solution is freeze-dried.

The deprotected polymer is analyzed by ¹H NMR; the spectrum confirms the total disappearance of the isopropylidene groups on the saccharide units.

### EXAMPLE 10

### Coupling of ODN with the Deprotected Saccharide Grafts:

Tests for coupling a hepatitis C virus nucleotide sequence (ODN 1) with the deprotected polyGVE were performed, under various conditions described below.

Nucleotide Sequence:

SEQ ID No. 1: 5′TCA-ATC-TCG-GGA-ATC-TCA-ATGTTA-G-3′

This sequence comprises a C₆—NH₂ coupling arm at the 5′ end as described in WO 91/19812.

This coupling reaction on the saccharide homopolymers is performed by reductive amination between the (masked) aldehyde function present on each saccharide unit (equilibrium of opening of the saccharide ring), and the primary amine function at the end of the 5′ amino arm of ODN.

Reaction scheme for the coupling of ODN with the deprotected PGVE:

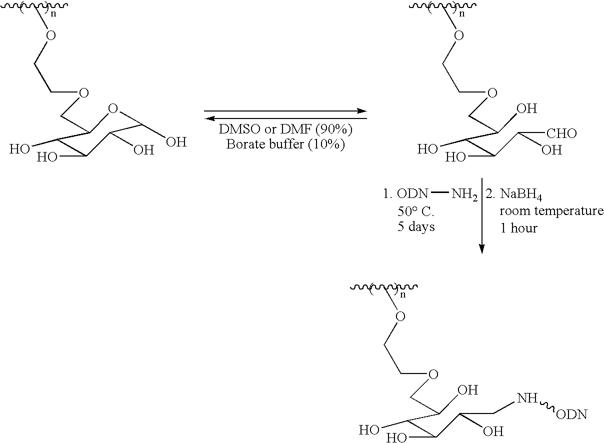

Test conditions:

organic solvent/aqueous buffer ratio: .90/10,

sodium borate buffer pH=9.3: 100 mmol.L⁻¹, 50 mmol.L⁻¹ or 25 mmol.L⁻¹,

organic solvent: DMF or DMSO,

temperature: 50° C.

1 ODN per 3 saccharide units, i.e. a theoretical maximum of 6 ODN/graft, given that the polyGVE grafts used have a degree of polymerization of 18.

Procedure:

For each sample, 5 nmol of ODN in 20 μL of aqueous buffer are introduced into 180 μL of an organic solution of deprotected polyGVE (FD33) (15 nmol of saccharide units). The mixture is left stirring at 50° C. for 5 days. The imine functions formed are reduced by adding NaBH₄ (three times 100 equivalents, at 1-hour intervals, at room temperature).

The samples are then dried on a speed-vac and then taken up in 200 μL of distilled water just before analysis by SEC (UH500 column, phosphate buffer eluent, pH=6.8 at 0.1 mol.L⁻¹).

Table Summarizing the Tests for Coupling ODN to the Deprotected PolyGVE FD33:

| Organic | Borate buffer | Coupling | Average number |
|---|---|---|---|
| solvent | pH 9.3 | yield | of ODN per graft |
| DMF | 25 mM | 20% | 1.2 |
| DMF | 50 mM | 20% | 1.2 |
| DMF | 100 mM | 20% | 1.2 |
| DMSO | 25 mM | 0 | 0 |
| DMSO | 50 mM | 0 | 0 |
| DMSO | 100 mM | 0 | 0 |

### EXAMPLE 11

### Terminal Functionalization of the PolyGVE Saccharide Grafts

Hydrazine Functionalization

In order to functionalize the aldehydeterminated polyGVEs with a hydrazine group, fluorenyl methyl carbazate (Fmoc) is used.

Terminal Functionalization of the Saccharide Grafts with a Hydrazine Function:

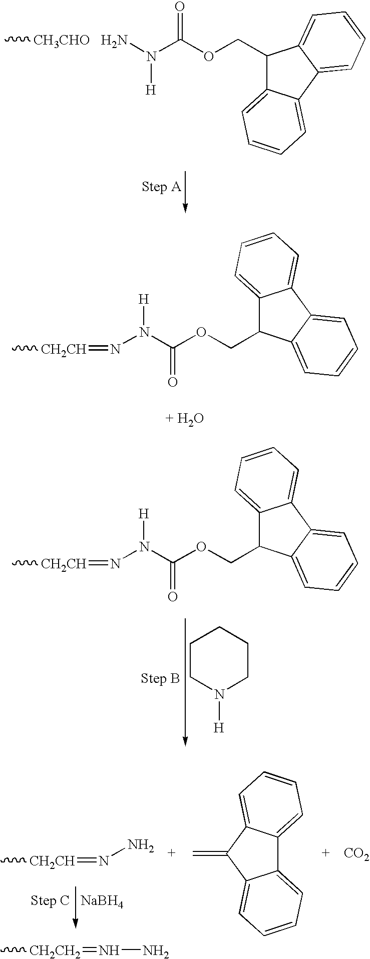

Step A:

A solution of polyGVE (1 g) in dichloromethane (sold by Aldrich, HPLC grade, reference 27,056-3) (4 mL) is placed under stirring and under nitrogen at room temperature. One equivalent of Fmoc (sold by Fluka, reference 46917) (0.125 g) dissolved in 2 mL of dichloromethane is added. After two hours, the polymer is precipitated in pentane.

The cloudy suspension is centrifuged until a clear filtrate is obtained. The pellet and the filtrate are derived under vane-pump vacuum. The polymer is analyzed by ¹H NMR and by MALDI-TOF mass spectrometry. These analyses indicate a quantitative functionalization of the grafts.

Step B:

polyGVE-Fmoc (0.600 g) is dissolved in 3 mL of dichloromethane. 1 mL of a solution of piperidine (sold by Aldrich, reference 10,409-4) in dry dichloromethane (0.5 mol.L⁻¹, 4 equivalents of piperidine per 1 equivalent of polymer) is added.

The mixture is stirred under nitrogen at room temperature for 1 hour 30 minutes. The polymer is precipitated in pentane and recovered by centrifugation. ¹H NMR analysis confirms the total disappearance of the Fmoc group.

Step C:

In order to reduce the hydrazone function formed at the end of the grafts, an excess of NaBH₄ (0.033 g, sold by Aldrich, reference 48,088-6) is introduced into 8 mL of a solution of the above polymer in dichloromethane in the presence of 1 mL of ethanol.

After reaction for five hours at room temperature, the reaction medium is diluted with CH₂Cl₂ and the excess NaBH₄ is hydrolyzed with saturated aqueous NaCl solution. As soon as this solution is added, an emulsion forms. After separation of the phases for thirty minutes, two clear phases are obtained.

The organic phase is washed three times with distilled water, dried over MgSO₄ and concentrated. The polymer is dried under vane-pump vacuum.

Amine Functionalization:

In order to functionalize the aldehydeterminated polyGVEs with a primary amine group, hexamethylenediamine (HMDA, sold by Aldrich, reference H1,169-6) is introduced at the end of the grafts by reductive amination.

Terminal functionalization of the saccharide grafts with an amine function.

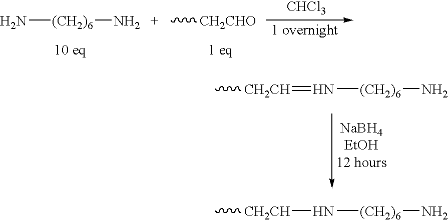

polyGVE (0.150 mg) is dissolved in chloroform (15 mL) and 0.057 mg of HMDA (10 equivalents) are added. The reaction mixture is stirred at room temperature for 12 hours.

In order to reduce the imines formed to secondary amines, NaBH₄ is introduced (10 equivalents in 1 mL of ethanol).

The excess NaBH₄ is hydrolyzed by adding 300 mL of saturated aqueous NH₄Cl solution.

The organic phase is extracted with 50 mL of chloroform, washed three times with 300 mL of saturated aqueous NH₄Cl solution and then once with saturated aqueous NaHCO₃ solution, dried over MgSO₄, concentrated and dried under vane-pump vacuum.

The analyses of the polymer by ¹H NMR and MALDI-TOF spectrometry confirm the structure of the functionalized grafts.

### EXAMPLE 12

### Attachment of the Saccharide Grafts to the Skeleton

One of the possibilities for obtaining the grafted structure is to react the grafts, functionalized with an amine function (or hydrazine), with the copolymer skeleton of NAM and NAS, as described below.

Reaction of the graft-NH₂ (or of the graft-NH—NH₂) with the activated ester functions of the NAM/NAS skeleton:

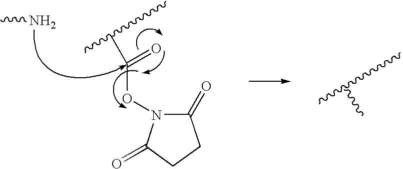

The graft-NH₂ (120 mg, 2×10⁻⁵ mol) and the skeleton (copolymer of NAM and NAS of 120,000 g/mol, NAM/NAS molar ratio equal to 60/40, 23 mg, 6×10⁻⁵ mol of NAS units) are dissolved under nitrogen in 2 mL of DMF in the presence of triethylamine (3 mg, 3×10⁻⁵ mol).

The mixture is placed under stirring (thermomixer) at 40° C. After five days, the solvent is removed by evaporation under vacuum. The residue is analyzed by SEC (eluent: DMF, polystyrene calibration).

The grafting yield is calculated by comparing the area of the peak corresponding to the residual grafts with the area of the peak corresponding to the grafts introduced using toluene as internal reference.

Under these conditions, a grafting yield of the order of 90% is obtained, i.e. an average of 72 grafts (side segments) per skeleton chain.

1. A biocompatible polymer with a molar mass of greater than 50,000 g/mol, preferably 90,000 g/mol, allowing the fixing of biological ligands, and comprising at least: a first linear segment consisting of a hydrophobic homopolymer resulting from the polymerization of a hydrophobic monomer A; a second linear segment consisting of a hydrophilic copolymer resulting from the copolymerization of a monomer B bearing a reactive function X and of a hydrophilic monomer C not bearing a reactive function, said second segment being covalently bonded to one end of the first segment and the two segments together constituting the skeleton of the polymer.

2. The polymer as claimed in claim 1, characterized in that the monomer A is chosen from methacrylate, acrylate, acrylamide, methacrylamide and styrene derivatives, preferably n-butyl acrylate, tert-butyl acrylate, tert-butylacrylamide, octadecylacrylamide or styrene.

3. The polymer as claimed in either of claims 1 and 2, characterized in that the monomer B is chosen from acrylate, methacrylate, acrylamide and methacrylamide functional derivatives and styrene functional derivatives, preferably N-acryloxysuccinimide, N-methacryloxysuccinimide, 2-hydroxyethyl methacrylate, 2-aminoethyl methacrylate, 2-hydroxyethyl acrylate, 2-aminoethyl acrylate or 1,2:3,4-di-O-isopropylidene-6-O-acryloyl-D-galactopyranose.

4. The polymer as claimed in any one of claims 1 to 3, characterized in that the monomer C is chosen from acrylamide, methacrylamide and N-vinylpyrrolidone derivatives, preferably N-vinylpyrrolidone or N-acryloylmorpholine.

5. The polymer as claimed in any one of claims 1 to 4, characterized in that X is chosen from amine and aldehyde functions and carboxylic acid functions activated in the form of N-hydroxysuccinimide ester.

6. The polymer as claimed in any one of claims 1 to 5, characterized in that the first segment has a molar mass of between 10,000 and 250,000 g/mol.

7. The polymer as claimed in any one of claims 1 to 6, characterized in that the second segment has a molar mass of greater than 40,000 g/mol and preferably greater than 80,000 g/mol.

8. The polymer as claimed in any one of claims 1 to 7, characterized in that the second segment is a random copolymer whose composition, expressed by the ratio of the amounts of monomers in moles: amount of monomer C to amount of monomer B, is between 1 and 10 and preferably between 1.5 and 4.

9. The polymer as claimed in any one of claims 1 to 8, also comprising at least one “side” segment consisting of a linear homopolymer resulting from the polymerization of a monomer D bearing a reactive function Y, said side segment being covalently bonded to the second segment at a single bonding point via reactive functions X of the monomer B.

10. The polymer as claimed in claim 9, characterized in that the reactive function Y is different than the reactive function X.

11. The polymer as claimed in claim 9, characterized in that the reactive function Y is identical to the reactive function X.

12. The polymer as claimed in claim 11, characterized in that the reactive functions X and Y are protected functions.

13. The polymer as claimed in any one of claims 9 to 12, characterized in that the monomer D is chosen from sugar derivatives, advantageously from galactose derivatives, and the monomer D is preferably 1,2:3,4-di-O-isopropylidene-6-O-(2-vinyloxyethyl)-D-galactopyranose.

14. The polymer as claimed in any one of claims 9 to 12, characterized in that the monomer D is chloroethyl vinyl ether.

15. The polymer as claimed in any one of claims 9 to 14, characterized in that the side segment has a molar mass of greater than 1500 g/mol.

16. The polymer as claimed in any one of claims 1 to 15, also comprising a “spacer” segment covalently intercalated between the first segment and the second segment, consisting of a linear homopolymer resulting from the polymerization of a hydrophilic monomer E, said monomer not bearing any reactive functions.

17. The polymer as claimed in claim 16, characterized in that the monomer E is chosen from acrylamide derivatives, methacrylamide derivatives, N-vinylpyrrolidone and N-acryloylmorpholine.

18. The polymer as claimed in either of claims 16 and 17, characterized in that the monomer E is identical to the monomer C.

19. A polymer-biological ligand conjugate comprising at least one biological ligand fixed to a polymer as defined in any one of claims 1 to 18.

20. The polymer-biological ligand conjugate as claimed in claim 19, characterized in that the biological ligand is fixed to the polymer directly by covalent coupling.

21. The polymer-biological ligand conjugate as claimed in claim 19, characterized in that the biological ligand is fixed to the polymer indirectly by a noncovalent interaction.

22. A device for capturing a target molecule with the aim of detecting it and/or assaying it and/or purifying it, comprising a solid support on which is immobilized a polymer-biological ligand conjugate as defined in any one of claims 19 to 21.

23. The device as claimed in claim 22, characterized in that the polymer-biological ligand conjugate is immobilized on the solid support by adsorption.

24. The device as claimed in claim 22, characterized in that the polymer-biological ligand conjugate is immobilized on the solid support by covalent bonding.

25. The device as claimed in any one of claims 22 to 24, characterized in that the biological ligand is capable of forming a ligand/antiligand capture complex.

26. The device as claimed in claim 25, characterized in that said antiligand constitutes the target molecule.

27. A process for synthesizing a polymer as claimed in any one of claims 1 to 18, characterized in that the linear skeleton of the polymer is prepared by growing chains by the reversible addition/fragmentation chain-transfer (RAFT) technique in the presence of a transfer agent of dithioester type.

28. A process for synthesizing a polymer as claimed in any one of claims 9 to 18, comprising the following steps:
the linear skeleton of the polymer is prepared by the reversible addition/fragmentation chain-transfer (RAFT) technique in the presence of a transfer agent of dithioester type,
the side segment is prepared independently by means of a controlled polymerization technique chosen from the techniques comprising living cationic polymerization, living anionic polymerization and reversible addition/fragmentation chain-transfer (RAFT) polymerization, and a reactive function capable of reacting with the reactive function X of the monomer B present on the skeleton is then introduced onto said side segment, at one end,
the linear skeleton and the side segment are placed in contact to allow the coupling.
